(19) United States
(12) Patent Application Publication
(10) Pub. No.: US 2025/0022601 A1
(43) Pub. Date: Jan. 16, 2025

(54) METHOD TO PREDICT AND DETECT ONSET OF DISEASES IN ORGANISMS

(71) Applicant: Shubham CHANDRA, Katy, TX (US)

(72) Inventor: Shubham CHANDRA, Katy, TX (US)

(21) Appl. No.: 18/221,779

(22) Filed: Jul. 13, 2023

(51) Int. Cl. G16H 50/30; G06Q 50/02; G06T 7/00; G06T 7/90; G16B 5/00; G16B 40/30; G16H 30/40

(52) U.S. Cl. CPC G16H 50/30; G06Q 50/02; G06T 7/0012; G06T 7/90; G16B 5/00; G16B 40/30; G16H 30/40

(57) ABSTRACT

Disease outbreaks pose significant threats to the health and productivity of both plants and organisms, with potential economic and ecological consequences. Timely identification and prediction of disease onset are crucial for implementing effective mitigation strategies. This abstract presents a novel method that combines clustering techniques with an AI framework based on CILEAB to predict the onset of diseases in plants and organisms.

The proposed method leverages unsupervised clustering algorithms to identify patterns and groupings within a dataset consisting of various disease-related parameters, such as environmental factors, genetic information, and symptom progression data. By clustering similar instances together, the method aims to capture shared characteristics among samples that could indicate the presence of an underlying disease.

### BACKGROUND

### Technical Field

The present disclosure relates to a method and computer-based apparatuses that can be used to detect the onset of diseases in organisms including humans and plants. In lieu of this patent application and research, no federally/Government sponsored research or development-based funding was used.

### Technical Background

Disease Detection is the process of identifying and assessing the presence of a disease in a plant or human. It is an important part of plant and human health management, as it can help to prevent the spread of disease, improve the effectiveness of treatment, and reduce the risk of death.

In agriculture, plant disease detection plays a crucial role by enabling early identification and intervention to prevent the spread of diseases and minimize crop losses. Traditionally, plant disease detection relied on visual inspection by human experts, which can be time-consuming, subjective, and limited by the expertise of the observer. However, advancements in technology have led to the development of various automated and non-destructive techniques for plant disease detection.

Here are some of the different technologies available:

Image-based approaches use digital images of plants or plant parts to detect diseases. These images can be captured using cameras or sensors, and various computer vision and image processing techniques are employed for analysis. Features such as color, texture, shape, and size are extracted from the images to classify and detect diseases.

Spectral imaging involves capturing and analyzing the electromagnetic radiation reflected or emitted by plants at different wavelengths. Hyperspectral imaging, for example, captures images across numerous narrow and contiguous bands of the electromagnetic spectrum. By analyzing the spectral signatures, diseases can be identified based on characteristic variations in the reflectance or emission patterns.

Sensor-based approaches use various types of sensors to detect plant diseases. For instance, infrared sensors can detect changes in plant temperature associated with disease infection. Gas sensors can detect volatile organic compounds released by infected plants, which can indicate the presence of diseases. Other sensors can measure parameters such as humidity, moisture, or electrical conductivity to infer disease conditions.

DNA-based methods involve molecular techniques for the detection of pathogens or disease-causing organisms in plants. Polymerase chain reaction (PCR) and its variants are commonly used to amplify and detect specific DNA sequences associated with pathogens. Other techniques such as DNA microarrays and next-generation sequencing are also employed for high-throughput detection and identification of pathogens.

Machine learning and artificial intelligence (AI) techniques are utilized in combination with various data sources, such as images, sensor readings, or molecular data, to develop predictive models for disease detection. These models learn patterns and relationships from the data and can make accurate predictions or classifications of plant diseases.

Hyperspectral remote sensing techniques involve the use of airborne or satellite-based sensors to collect high-resolution spectral data of large agricultural areas. These techniques can detect subtle changes in plant health and identify disease outbreaks by analyzing the spectral signatures of crops.

Internet of Things (IoT) and sensor networks utilize networks of sensors deployed in agricultural fields to monitor various environmental parameters, such as temperature, humidity, soil moisture, and leaf wetness. By integrating this sensor data with disease models or machine learning algorithms, early signs of diseases can be detected and alerts can be generated for farmers.

These new technologies have the potential to revolutionize plant disease detection and management. By enabling early detection and intervention, they can help to reduce crop losses and improve agricultural productivity.

The choice of technology for disease detection and management in agriculture depends on a number of factors, including the specific disease, crop type, available resources, and the desired level of accuracy and automation. Often, a combination of techniques may be employed to improve disease detection and management strategies.

Global Positioning System (GPS)

GPS can be used in disease detection in a number of ways, including:

Geospatial Data Collection: GPS can be used to collect geospatial data, such as the coordinates and timestamps of disease-related events or samples. This data can be used to map disease distribution patterns and identify high-risk areas.

Spatial Analysis: GPS data can be integrated with other spatial information, such as satellite imagery or geographic information systems (GIS), to conduct spatial analysis of disease patterns. This can help to identify spatial relationships, hotspots, or environmental risk factors associated with the disease.

Disease Surveillance: GPS can support disease surveillance efforts by enabling real-time data collection and reporting. This information can be used for early detection, monitoring disease trends, and informing public health interventions.

It is important to note that GPS technology can provide valuable spatial data for disease surveillance and tracking, but it is typically used in combination with other tools and methods, such as laboratory testing, epidemiological investigations, and data analysis techniques. The integration of GPS data with other sources of health information enhances the understanding of disease dynamics and facilitates more effective disease prevention and control strategies.

In human disease detection, healthcare professionals collect a patient's medical history, including symptoms, family history, and lifestyle factors. Physical examinations are conducted to observe visible signs and symptoms, such as skin abnormalities, swelling, or abnormal sounds.

Laboratory Testing: Various laboratory tests are used to aid in disease diagnosis. These include blood tests, urine tests, tissue biopsies, imaging tests (X-rays, CT scans, MRIs), and molecular tests (PCR, gene sequencing). These tests help identify pathogens, measure specific biomarkers, or detect abnormal cell growth.

Medical Imaging: Imaging techniques like X-rays, CT scans, MRI, ultrasound, or PET scans are used to visualize internal structures, organs, and abnormalities within the body. These images help in detecting tumors, infections, fractures, or other structural changes associated with diseases.

Biomarker Analysis: Analysis of specific biomarkers, such as proteins, hormones, enzymes, or genetic markers, can aid in disease detection. Blood tests, for example, can measure levels of specific markers to identify diseases or monitor treatment responses.

In both plants and humans, advancements in technology, such as artificial intelligence (AI), machine learning, and big data analytics, are increasingly being employed to improve disease detection accuracy and efficiency. These technologies enable the development of automated systems for faster and more precise analysis of symptoms, images, or molecular data, facilitating early detection and timely interventions.

Disease detection poses several challenges due to the complex nature of organisms' diseases and the diverse factors that can influence their detection. Some of the key challenges include:

Symptom Variability: Diseases can exhibit a wide range of symptoms, and these symptoms can vary depending on the plant species, the stage of disease progression, environmental conditions, and pathogen strains. This variability makes it challenging to develop universal detection methods that can accurately identify all types of diseases across different species.

Similar Symptoms: Certain diseases may exhibit symptoms that resemble those caused by other factors, such as nutrient deficiencies, environmental stress, or physical damage. Distinguishing between disease symptoms and non-disease-related symptoms can be difficult, requiring expert knowledge and careful analysis.

Pathogen Diversity: Diseases can be caused by a multitude of pathogens, including bacteria, fungi, viruses, and nematodes. Each pathogen has its own unique characteristics, life cycle, and infection patterns, making it challenging to develop generalized detection methods that can effectively detect all types of pathogens.

Early Detection: Early detection is crucial for effective disease management. However, in many cases, diseases remain undetected until visible symptoms appear, by which time significant damage may have already occurred. Developing techniques for early detection, such as molecular assays or remote sensing technologies, is a challenge that requires sensitive and specific detection methods.

Cost and Accessibility: Some disease detection methods, particularly those involving advanced laboratory techniques or specialized equipment, can be costly and require technical expertise. Making disease detection methods affordable, accessible, and user-friendly for farmers, medical researchers, and agronomists is an ongoing challenge.

Addressing these challenges requires interdisciplinary research, collaboration among scientists and industry experts, advancements in technology, and the integration of different detection approaches. Improving disease detection methods will help in early intervention, effective disease management, and sustainable practices.

Predicting the onset of diseases in plants/organisms using AI models presents several challenges that researchers and practitioners need to address. Some of the major challenges include:
1. Limited and unbalanced training data: AI models require large and diverse datasets for effective training. However, obtaining labeled data for plant diseases can be challenging due to limited availability and the need for expert annotation. Moreover, the distribution of data among different disease classes may be unbalanced, making it difficult for the model to generalize well across all diseases.
2. Variability in disease symptoms: Plant diseases can exhibit a wide range of symptoms, including variations in color, texture, shape, and size. Capturing and representing this variability in training data can be complex. AI models must be trained to recognize and differentiate between subtle disease symptoms and other factors that might affect plant appearance, such as nutrient deficiencies, environmental stress, or physical damage.
3. Transferability to new environments: Models trained on data from specific geographic regions or plant species may struggle to generalize well when deployed in new environments or on different plant varieties. The variability in environmental conditions, plant genetics, and disease prevalence requires robust models that can adapt and generalize across diverse settings.
4. Early detection and false positives: Detecting diseases in their early stages is crucial for effective disease management. However, early symptoms may be subtle and easily missed, leading to delayed or inaccurate predictions. AI models need to be sensitive enough to identify these early indicators while minimizing false positives, which can result in unnecessary interventions or increased costs.
5. Scalability and real-time processing: Deploying AI models for large-scale agricultural applications requires efficient processing and scalability. Real-time or near real-time prediction is crucial to enable timely interventions. Balancing the computational demands of AI algorithms with the need for rapid analysis poses a challenge, especially when dealing with high-resolution images or large datasets.
6. Interpretability and trust: AI models are often considered black boxes, making it challenging to understand the reasoning behind their predictions. In the agricultural domain, it is crucial to have transparent and interpretable models to gain trust and facilitate decision-making. Ensuring explainability and interpretability of AI models is important to gain acceptance and adoption in plant disease prediction.

From an image analysis perspective, CIELAB, also known as LAB color space or Lab* color space, is a color model used to represent colors in a device-independent and perceptually uniform manner. It was developed by the International Commission on Illumination (CIE) as a standard color space for accurately describing and comparing colors.

The CIELAB color model consists of three components:

L* (Lightness): The L* component represents the perceived lightness or brightness of a color. It ranges from0 to 100, where 0 represents black and 100 represents white. The midpoint of the scale, L*=50, is considered a neutral gray.

a* (Green-Red axis): The a* component represents the position along the green-red axis. Positive values indicate a shift towards red, while negative values indicate a shift towards green. The range of a* typically extends from −128 to +127.

b* (Blue-Yellow axis): The b* component represents the position along the blue-yellow axis. Positive values indicate a shift towards yellow, while negative values indicate a shift towards blue. The range of b* typically extends from −128 to +127.

The CIELAB color space is designed to be perceptually uniform, meaning that an equal numerical difference in the Lab* values corresponds to a similar perceptual difference in color across the entire color space. This makes it useful for various color-related applications, such as color management, color matching, and color difference calculations.

Clustering can be used to analyze CIELAB data by grouping similar colors together based on their perceptual similarities in the LAB color space. Clustering is a technique used in unsupervised machine learning to group similar data points together based on their inherent similarities or patterns. The objective is to discover natural groupings or clusters within the data, where data points within the same cluster are more similar to each other compared to those in other clusters. Clustering is widely used in various domains, including data analysis, pattern recognition, image processing, and customer segmentation.

Different types of clustering algorithms exist, and they can be categorized based on various factors, such as the underlying algorithmic approach, the shape of the clusters, or the assumptions made during the clustering process. Here are some commonly used types of clustering: K-Means Clustering: It is a centroid-based clustering algorithm. It aims to partition the data into K clusters, where K is a predetermined number. The algorithm iteratively assigns data points to the nearest cluster centroid and updates the centroids until convergence. K-Means assumes that clusters are spherical and have similar sizes.

Hierarchical Clustering: This type of clustering creates a hierarchy of clusters using a bottom-up (agglomerative) or top-down (divisive) approach. In agglomerative hierarchical clustering, each data point initially represents a single cluster, and then clusters are successively merged based on their similarity until a single cluster containing all the data points is formed. Divisive hierarchical clustering works in the opposite way, starting with a single cluster and iteratively splitting it into smaller clusters. The result is a tree-like structure called a dendrogram that shows the relationship between the clusters at different levels.

Density-Based Clustering: Density-based clustering algorithms, such as DBSCAN (Density-Based Spatial Clustering of Applications with Noise), group together data points that are densely connected to each other. It identifies clusters as regions of high-density separated by regions of low-density. DBSCAN does not require a predetermined number of clusters and can discover clusters of arbitrary shapes.

Gaussian Mixture Models (GMM): GMM is a probabilistic model that assumes the data points are generated from a mixture of Gaussian distributions. The algorithm aims to estimate the parameters of these distributions and assign data points to the most likely cluster based on their probabilities. GMM can handle data with complex distributions and can identify clusters with different shapes and sizes.

Fuzzy Clustering: Fuzzy clustering allows for the partial membership of data points to multiple clusters. Instead of hard assignments, fuzzy clustering assigns membership values to data points, indicating the degree of association with each cluster. Fuzzy C-Means (FCM) is a well-known fuzzy clustering algorithm.

Model-Based Clustering: Model-based clustering algorithms, such as Expectation-Maximization (EM), assume that the data is generated from a specific statistical model. These algorithms estimate the parameters of the model and assign data points to clusters based on their likelihood under the model. The number of clusters may be automatically determined using techniques like the Bayesian Information Criterion (BIC).

### SUMMARY

In accordance with the purpose(s) of the invention, as embodied and broadly described herein, this disclosure, in one aspect, relates to image-based approach for predicting the onset of diseases in organisms, including plants, humans, and animals, is the application of CIELAB and machine vision techniques. These methods leverage the power to analyze images and extract meaningful patterns or features that are indicative of disease conditions.

In the case of plant diseases, the current invention has developed an image-based disease detection systems—wherein the models are trained on large datasets of labeled images of healthy and diseased plants to learn to differentiate between different disease symptoms. The trained model can then be used to classify and predict the presence of diseases in new plant images.

For human and animal diseases, similar approaches are being employed. Medical imaging, such as X-rays, CT scans, MRI scans, and histopathological images, can be analyzed using the learning algorithms to detect and predict diseases.

In the present invention, a method to predict the onset of diseases in plants and animals has been developed that works by combining clustering techniques with CIELAB color space analysis. K-means clustering algorithms, groups similar samples based on their disease status or symptoms. By applying this algorithm to labeled datasets of healthy and diseased samples, distinct clusters representing different disease stages or conditions have been identified. In parallel, the CIELAB color space can be used to extract color-related features from images of plants/fruits/leaves or organisms. CIELAB provides a perceptually uniform color space, enabling more accurate representation of color differences. By quantifying the color information in images using CIELAB color values, specific patterns or changes related to disease progression are captured. Combining clustering with CIELAB analysis, the collected color data is being used to identify and associate clusters of samples with specific disease stages. This allows for the development of a predictive model that can determine the likelihood of disease onset based on the color patterns observed in new samples. By monitoring and comparing color changes over time, these models can aid in early detection and provide valuable insights for disease management and intervention. The CIELAB color space, also referred to as L*a*b*, is a color space defined by the International Commission on Illumination in 1976. It expresses color as three values: L* for perceptual lightness, and a* and b* for the four unique colors of human vision: red, green, blue, and yellow. CIELAB was intended as a perceptually uniform space, where a given numerical change corresponds to a similar perceived change in color.

The important thing for the CIELAB color space is that it is device-independent, “standard observer” model. The colors it defines are not relative to any particular device such as a computer monitor or a printer, but instead relate to the CIE standard observer which is an averaging of the results of color matching experiments under laboratory conditions.

The CIELAB space is three-dimensional, and covers the entire range of human color perception, or gamut. It is based on the opponent color model of human vision, where red and green form an opponent pair, and blue and yellow form an opponent pair. The lightness value, L*, also referred to as “Lstar,” defines black at 0 and white at 100. The a* axis is relative to the green-red opponent colors, with negative values toward green and positive values toward red. The b* axis represents the blue-yellow opponents, with negative numbers toward blue and positive toward yellow. The a* and b* axes are unbounded and depending on the reference white they can easily exceed ±150 to cover the human gamut. Nevertheless, software implementations often clamp these values for practical reasons. For instance, if integer math is being used it is common to clamp a* and b* in the range of −128 to 127.

The hue of a color is quantified by its hue angle h_{ab} in the a*b*-plane, given in degrees) (°). The hue angle of a color can be calculated from the color coordinates:

$${h}_{ab} = arctan ( \frac{{b}^{*}}{{a}^{*}} ) . \qquad Equation 1$$

Chroma is the amount of saturation of a color. Colors of high chroma are said to be clear, bright or brilliant. Dull (pastel) colors have a low chroma.

$${C}_{ab} = \sqrt{{a}^{{*}^{2}} + {b}^{{*}^{2}}} . \qquad Equation 2$$

The method is validated in a MATLAB based application. This documents the steps to be followed within the database. There are two elements to this method, first one is the training side, wherein the following steps are followed:
STEP 1. Identify the crop/plant and the specific disease type to be identified. To analyze the photo/s of the specific diseased crop/plant, draw a Region of Interest (ROI) around the diseased parts of the plant image.
STEP 2. After completing the ROI for one image, click “Next” to move on to the next image.
STEP 3. Repeat this process for all the images in the set.
STEP 4. Finally, for the training process all images from the set are read and converted to the CIELAB color space.
STEP 5. L, a, and b values are extracted from the drawn ROIs in each image.
STEP 6. Any zero values are removed, and the remaining values are adjusted to ensure they have the same data length.
STEP 7. The a values are then clustered into two groups. The group with the majority of values is retained, while the other group is discarded.
STEP 8. Index values for the clustered a group are determined, and corresponding 1 and b values are extracted.
STEP 9. The final extracted values are further clustered into ten groups, and ranges for each group are calculated and saved to disk. This data is saved in an excel file.

The second element is to analyze an image for a selected product, follow these steps:
STEP 1. Choose the product name and select an image for analysis.
STEP 2. Use the slider to remove any unnecessary background.
STEP 3. Click the “Detect” button.
STEP 4. The segmented image is converted to the CIELAB color space.
STEP 5. L, a, and b values are extracted from the image.
STEP 6. Zero values are eliminated from the extracted values.
STEP 7. The L, a, and b values are clustered into three groups.
STEP 8. The group with the highest number of values is discarded, and the other two clusters are merged. This resulting cluster contains the pixel values of the image.

From the database folder:
STEP 9. Extract the names of all excel files.
STEP 10. Read the excel file that matches the product name in the app.
STEP 11. Extract all sheet names (disease names) from this XLS file.
STEP 12. Read the data for each sheet one by one.
STEP 13. Extract the L, A, and B ranges from the XLS file.
STEP 14. For each pixel: Check if the 1, a, and b values fall within any of the corresponding ranges.
STEP 15. Mark a match as 1 and no match as 0.
STEP 16. Calculate the percentage of matches by counting the total number of 1 values and dividing it by the total number of 1 and 0 values.
STEP 17. Repeat the above process for other sheets (diseases):
STEP 18. Save the percentage of matches for 1, a, and b values.
STEP 19. Assign weights to the A percentage (50%), B percentage (35%), and L percentage (15%).
STEP 20. Sum up the weighted percentages to obtain a total overall percentage for each disease.
STEP 21. The disease with the highest percentage value is identified and displayed in the app.

By following these steps, the analysis determines the most likely disease based on the image's 1, a, and b values, providing relevant disease information in the app.

### BRIEF DESCRIPTION OF THE DRAWINGS

Reference is made to the accompanying drawings in which is shown an illustrative embodiment of the invention, from which its novel features and advantages will be apparent.

FIG. 1 is a simplified illustration of the Method to predict the on-set of diseases in plants and other organisms.

FIG. 2a is a simple illustration of the training method for the development of the training matrix. FIG. 2b is an illustration of the selection of the Regions of Interest to develop the relevant matrix.

FIG. 3 is the schematics of the analysis used to predict the disease for the evaluated leaf sample.

FIG. 4 is an image of an apple leaf that has been infected with scab.

FIG. 5 is an image of an apple leaf that has been infected with black rot. FIG. 6 is an image of an apple leaf that has been infected with cedar apple rust.

### DESCRIPTION OF THE PREFERRED EMBODIMENTS

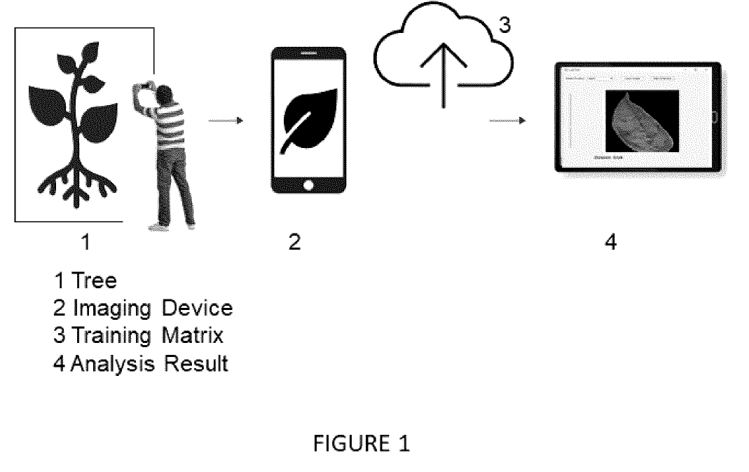

Referring to FIG. 1, it will be seen that an illustrative includes there is plant/tree/organisms that is being photographed using an imaging device 2. 3 is a cloud serve based training matrix that consists of CIELAB data for diseased plants/organisms. 4 is the analysis that is conducted, by comparing the CILEAB data for the plant with the 3 matrix, and documents the disease the plant is infected with.

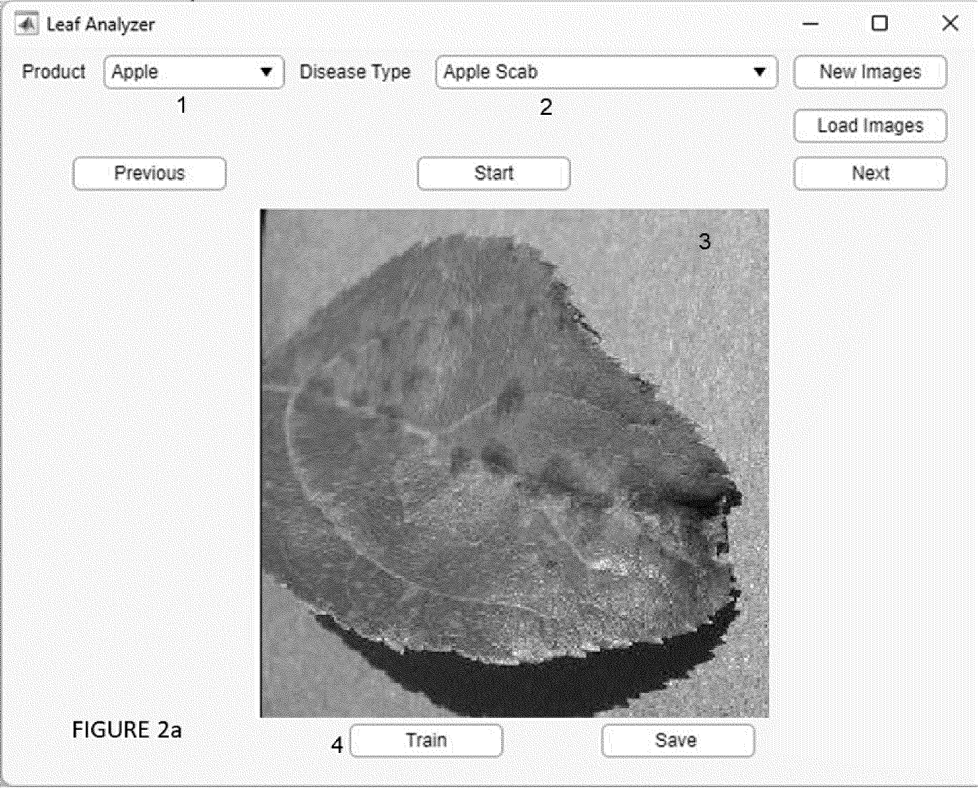

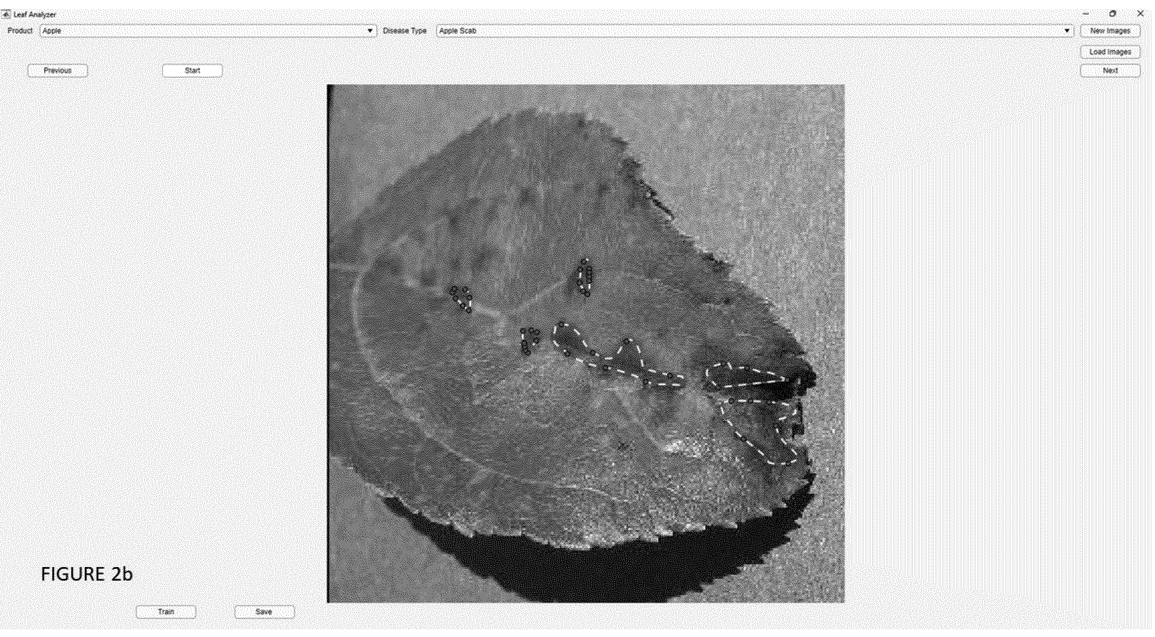

Referring to FIG. 2, it will be seen that a schematic that includes 1 the kind of product, 2, the kind of disease, 3 the image of the leaf/plant/organism, and 4 is the button to develop a training database based upon it.

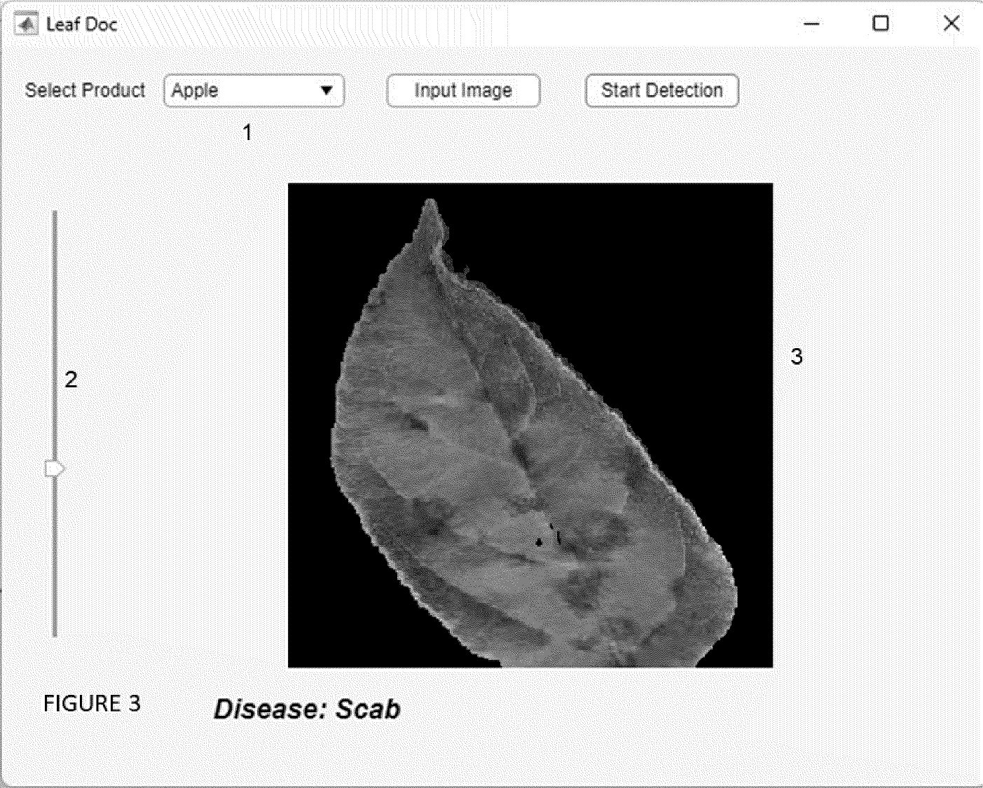

Referring to FIG. 3, it will be seen that an illustrative includes 1 the kind of product, 2, the slider to select for removal of the background/noise, 3 the image of the leaf/plant/organism to be evaluated for.

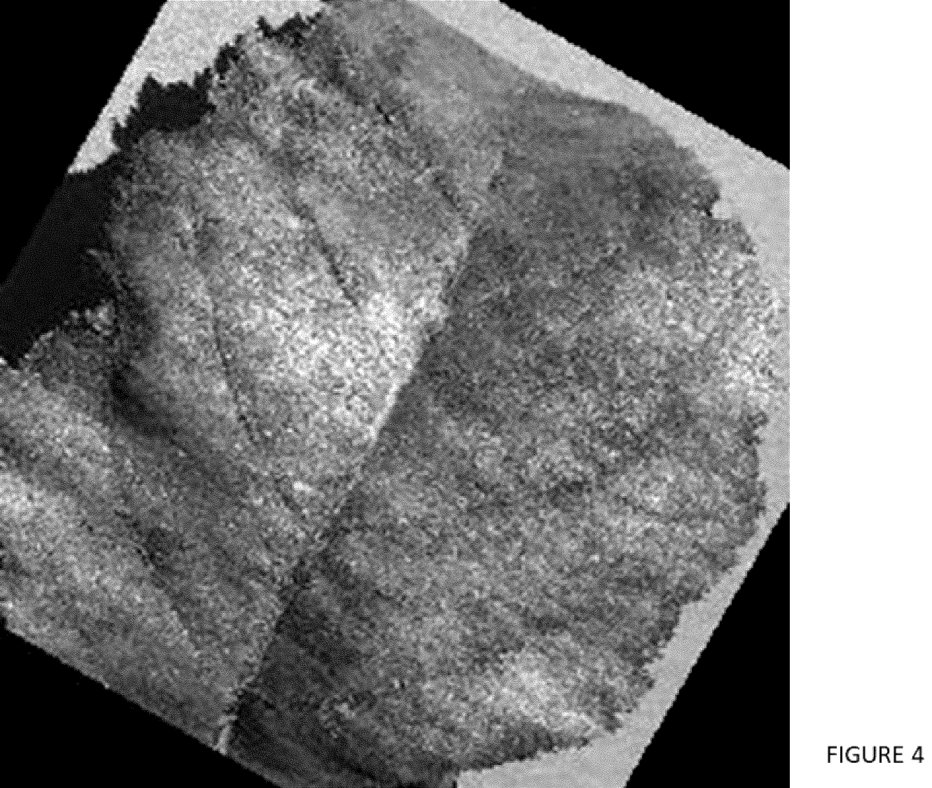

Referring to FIG. 4, it is photo of an apple leaf infected with scab disease.

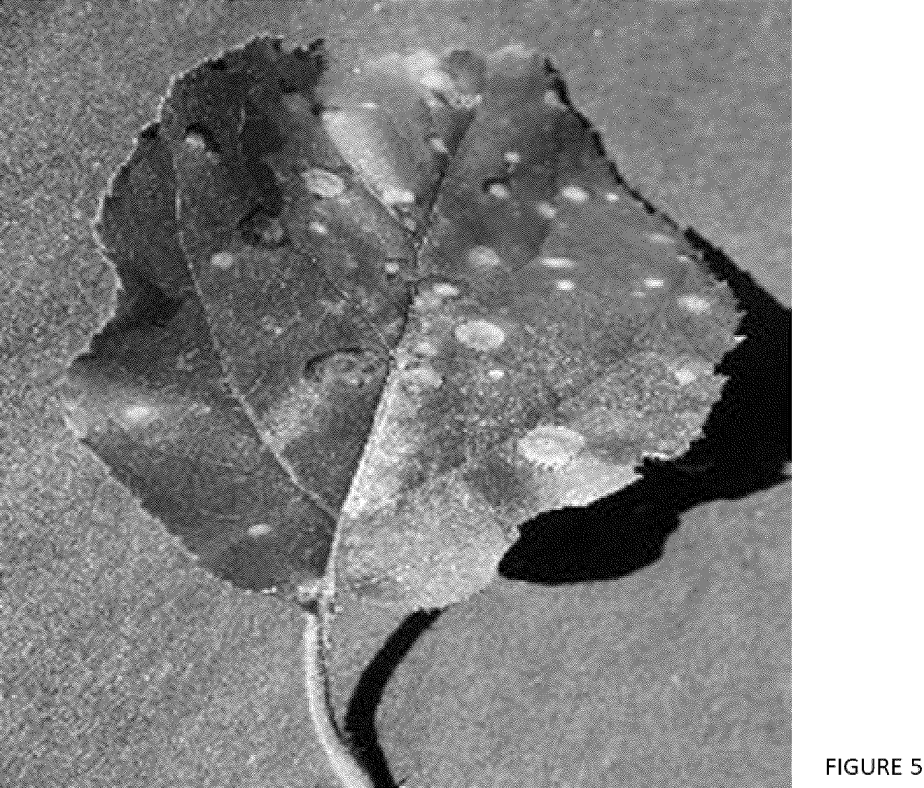

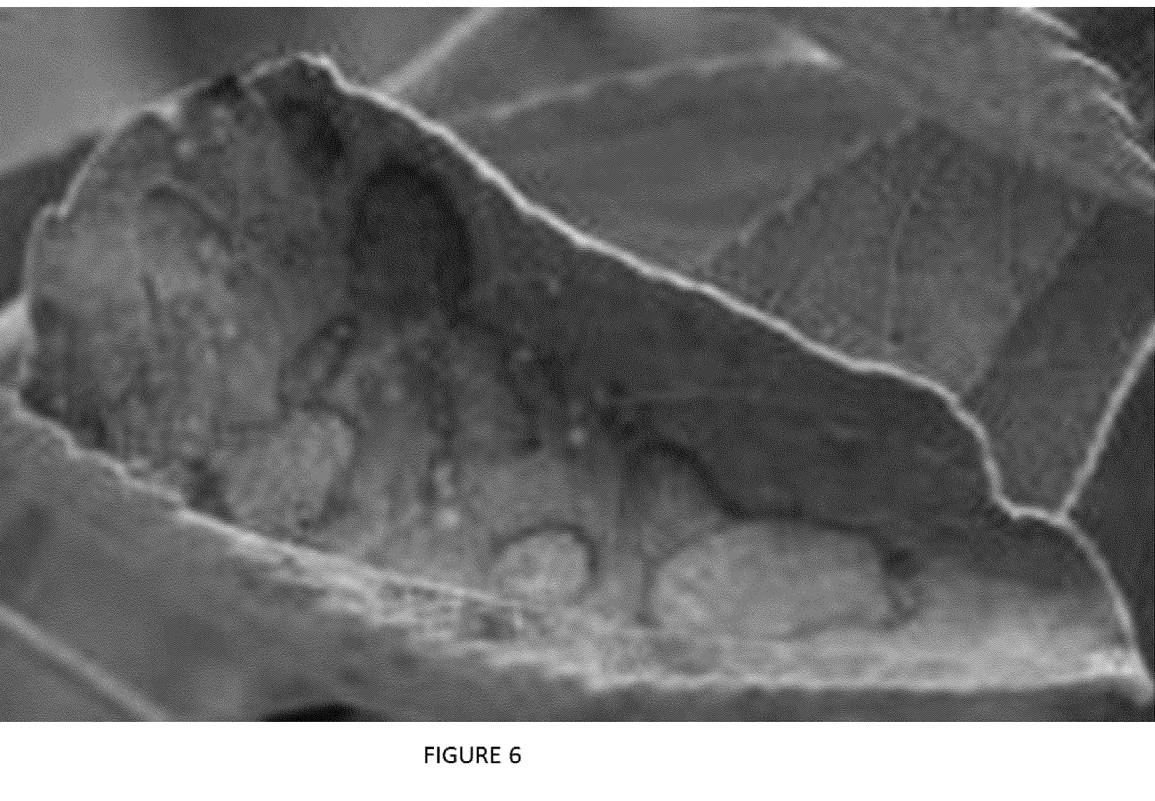

Referring to FIG. 5, it is photo of an apple leaf infected with black rot.

Referring to FIG. 6, it is photo of an apple leaf infected with cedar apple rust.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described.

As used in the specification and the appended claims, the singular forms “a,” “an” and “the” include plural referents unless the context clearly dictates otherwise.

### EXAMPLES

The invention will now be illustrated, but not limited, by reference to the specific embodiments described in the following examples.

### Example 1

This example documents the training module for the apple and the disease apple scab. After loading FIG. 4 and drawing Regions of Interest (ROI) on the diseased parts, the training process converts them to CIELAB space, and extracts the 1, a and b values from the drawn ROI. All zero values are removed first, and then these values are adjusted to minimum length so that all 1, a and b have same data length. Then the a values are clustered into 2 Groups, the group with most values is kept, and the other group is discarded. The index values of a are found, and the 1 and b values corresponding to these indexed values are extracted. The final extracted values are further clustered into 10 groups each and ranges for each group are found and saved to disk. The data is shown in Table 1.

TABLE1
CIELAB values in 10 Groups
for Apple Scab Disease

| | | min | max |
|---|---|---|---|
| I value | L_1 | 24.23 | 31.85 |
| | L_2 | 32.02 | 35.62 |
| | L_3 | 35.64 | 38.33 |
| | L_4 | 38.34 | 41.00 |
| | L_5 | 41.04 | 44.17 |
| | L_6 | 44.23 | 48.01 |
| | L_7 | 48.07 | 53.03 |
| | L_8 | 53.16 | 59.02 |
| | L_9 | 59.08 | 66.19 |
| | L_10 | 66.53 | 93.50 |
| a value | A_1 | −0.02 | 2.73 |
| | A_2 | 2.84 | 5.32 |
| | A_3 | 5.41 | 7.80 |
| | A_4 | 7.83 | 9.93 |
| | A_5 | 9.96 | 11.73 |
| | A_6 | 11.77 | 13.32 |
| | A_7 | 13.34 | 14.80 |
| | A_8 | 14.82 | 16.31 |
| | A_9 | 16.35 | 18.26 |
| | A_10 | 18.29 | 22.12 |
| b value | B_1 | 6.76 | 9.97 |
| | B_2 | 10.09 | 12.30 |
| | B_3 | 12.31 | 14.90 |
| | B_4 | 14.92 | 18.36 |
| | B_5 | 18.43 | 21.93 |
| | B_6 | 22.01 | 25.27 |
| | B_7 | 25.38 | 28.66 |
| | B_8 | 28.76 | 32.24 |
| | B_9 | 32.39 | 36.31 |
| | B_10 | 36.45 | 41.01 |

### Example 2

This example documents the training module for the apple and the disease apple black rot. After loading FIG. 5 and drawing Regions of Interest (ROI) on the diseased parts, the training process converts them to CIELAB space, and extracts the 1, a and b values from the drawn ROI. All zero values are removed first, and then these values are adjusted to minimum length so that all 1, a and b have same data length. Then the a values are clustered into 2 Groups, the group with most values is kept, and the other group is discarded. The index values of a are found, and the 1 and b values corresponding to these indexed values are extracted. The final extracted values are further clustered into 10 groups each and ranges for each group are found and saved to disk. The data is shown in Table 2.

TABLE 2
CIELAB values in 10 Groups
for Black Rot Disease

| | | min | max |
|---|---|---|---|
| | L_1 | 33.0 | 40.2 |
| I value | L_2 | 40.3 | 45.4 |
| | L_3 | 45.4 | 50.8 |
| | L_4 | 50.8 | 56.4 |
| | L_5 | 56.5 | 62.2 |
| | L_6 | 62.2 | 67.7 |
| | L_7 | 67.7 | 73.0 |
| | L_8 | 73.0 | 78.0 |
| | L_9 | 78.0 | 83.0 |
| | L_10 | 83.0 | 94.6 |
| | A_1 | 10.9 | 13.6 |
| a value | A_2 | 13.6 | 15.8 |
| | A_3 | 15.8 | 17.8 |
| | A_4 | 17.8 | 19.8 |
| | A_5 | 19.8 | 21.7 |
| | A_6 | 21.7 | 23.4 |
| | A_7 | 23.4 | 25.0 |
| | A_8 | 25.0 | 26.8 |
| | A_9 | 26.8 | 29.1 |
| | A_10 | 29.1 | 34.2 |
| | B_1 | 15.2 | 18.6 |
| b value | B_2 | 18.6 | 21.7 |
| | B_3 | 21.8 | 24.7 |
| | B_4 | 24.7 | 27.3 |
| | B_5 | 27.3 | 29.7 |
| | B_6 | 29.7 | 32.1 |
| | B_7 | 32.1 | 34.6 |
| | B_8 | 34.6 | 37.6 |
| | B_9 | 37.6 | 41.8 |
| | B_10 | 41.8 | 52.6 |

### Example 3

This example documents the training module for the apple and the disease apple cedar rust. After loading FIG. 6 and drawing Regions of Interest (ROI) on the diseased parts, the training process converts them to CIELAB space, and extracts the 1, a and b values from the drawn ROI. All zero values are removed first, and then these values are adjusted to minimum length so that all 1, a and b have same data length. Then the a values are clustered into 2 Groups, the group with most values is kept, and the other group is discarded. The index values of a are found, and the 1 and b values corresponding to these indexed values are extracted. The final extracted values are further clustered into 10 groups each and ranges for each group are found and saved to disk. The data is shown in Table 2.

TABLE 3
CIELAB values in 10 Groups
for Apple Cedar Rust Disease

| | | min | max |
|---|---|---|---|
| | L_1 | 11.6 | 18.9 |
| I value | L_2 | 20.6 | 29.1 |
| | L_3 | 29.6 | 37.5 |
| | L_4 | 38.4 | 44.4 |
| | L_5 | 45.2 | 51.3 |
| | L_6 | 52.4 | 57.9 |
| | L_7 | 58.5 | 64.7 |
| | L_8 | 65.2 | 71.3 |
| | L_9 | 71.7 | 77.3 |
| | L_10 | 79.4 | 81.6 |
| | A_1 | 11.6 | 14.9 |
| a value | A_2 | 15.1 | 18.2 |
| | A_3 | 18.4 | 21.2 |
| | A_4 | 21.5 | 23.9 |
| | A_5 | 24.2 | 27.0 |
| | A_6 | 27.3 | 30.4 |
| | A_7 | 30.6 | 33.7 |
| | A_8 | 34.0 | 36.3 |
| | A_9 | 38.5 | 41.6 |
| | A_10 | 44.4 | 48.0 |
| | B_1 | 10.2 | 13.1 |
| b value | B_2 | 17.8 | 22.8 |
| | B_3 | 24.9 | 31.5 |
| | B_4 | 32.2 | 38.7 |
| | B_5 | 40.3 | 46.4 |
| | B_6 | 47.1 | 52.8 |
| | B_7 | 54.2 | 59.0 |
| | B_8 | 59.4 | 63.6 |
| | B_9 | 64.3 | 68.7 |
| | B_10 | 69.2 | 75.4 |

What is claimed is:

1. A method for predicting the onset of diseases in plants using a CIELAB and clustering-based machine vision application, comprising the steps of:
a. Acquiring image data of plants, fruits or leaves, wherein the image data comprises visual features relevant to disease onset.
b. Preprocessing the acquired image data to enhance quality and extract relevant visual features.
c. Applying a clustering algorithm to the preprocessed image data to identify patterns and groupings based on the extracted visual features.
d. Utilizing a machine vision application based on the CIELAB color space and the clustered image data to generate predictive models for disease onset.
e. Training the machine vision application on the clustered image data to learn complex relationships between the visual features and the onset of diseases.
f. Receiving new instances of image data and preprocessing the new image data to extract relevant visual features.
g. Applying the trained machine vision application to the preprocessed new image data to predict the onset of diseases in plants.
h. Outputting the predicted disease onset information, facilitating early detection, and enabling proactive management strategies for disease control and mitigation efforts in plants.

2. A method for predicting the onset of diseases in humans including using a CIELAB and clustering-based machine vision application, comprising the steps of:
a. Acquiring image data of individuals or internal organs, wherein the image data comprises visual features relevant to disease onset.
b. Preprocessing the acquired image data to enhance quality and extract relevant visual features.
c. Applying a clustering algorithm to the preprocessed image data to identify patterns and groupings based on the extracted visual features.
d. Utilizing a machine vision application based on the CIELAB color space and the clustered image data to generate predictive models for disease onset.
e. Training the machine vision application on the clustered image data to learn complex relationships between the visual features and the onset of diseases.
f. Receiving new instances of image data and preprocessing the new image data to extract relevant visual features.
g. Applying the trained machine vision application to the preprocessed new image data to predict the onset of diseases in humans.
h. Outputting the predicted disease onset information, facilitating early detection, and enabling proactive management strategies for disease control and mitigation efforts in humans.

3. A method of claim 1, further configured to integrate into robotic arm, equipped with handgrip mechanism to allow for automated harvesting, pruning and disease application mitigation.

4. A method of claim 1, further configured to integrate into drone delivery system, equipped with handgrip mechanism to allow for automated harvesting, pruning and disease application mitigation.
